(19) United States
(12) Patent Application Publication
(10) Pub. No.: US 2014/0114126 A1
(43) Pub. Date: Apr. 24, 2014

(54) UNIVERSAL ENDOSCOPE ATTACHMENT SYSTEM AND RELATED METHODS OF USE

(71) Applicant: Boston Scientific Scimed, Inc., Maple Grove, MN (US)

(72) Inventor: Russell P. DRESHER, Hudson, MA (US)

(73) Assignee: Boston Scientific Scimed, Inc., Maple Grove, MN (US)

(21) Appl. No.: 14/057,884

(22) Filed: Oct. 18, 2013

Related U.S. Application Data
(60) Provisional application No. 61/717,438, filed on Oct. 23, 2012.

(51) Int. Cl. A61B 1/00; A61B 1/012

(52) U.S. Cl. CPC A61B 1/00112; A61B 1/0125; A61B 1/00082; A61B 1/00066
USPC 600/106

(57) ABSTRACT

A device for releasable coupling of a first medical tool to a second medical tool. The device may include an elongate member having a first end and a second end. The first end may be configured to be secured to a portion of the first medical tool, and the second end may be secured to a coupling sleeve. The coupling sleeve defines a lumen that may receive a portion of the second medical tool.

### CROSS-REFERENCE TO RELATED APPLICATIONS

This application claims the benefit of priority from U.S. Provisional Application No. 61/717,438 filed on Oct. 23, 2012, the entirety of which is incorporated by reference herein.

### FIELD

Embodiments of the present disclosure relate generally to endoscopic medical devices suitable for use in medical procedures, and more particularly to systems for operatively coupling multiple endoscopic devices.

### BACKGROUND

Minimally invasive medical procedures utilize instruments such as an endoscopic device to, among other things, visualize a patient's internal cavities and organs and/or perform procedures within the patient. An endoscopic device is inserted into a patient's body through a natural opening or through a percutaneous incision, and such devices typically include a hollow steerable flexible tube with one or more channels to deliver therapy, illumination, visualization, irrigation, or aspiration. The diameter of the endoscopic tube generally depends on the lumens or organs being observed.

Endoscopes provide mechanical support to a variety of medical devices, which themselves cannot be directly passed through bodily lumens due to their fragility and lack of flexibility. Such medical devices may include optical devices, such as cameras or illuminators, or surgical instruments such as forceps, snares, scissors, or smaller endoscopes. Relatively small endoscopes can be passed through the working channel of a larger endoscope.

Procedures such as retrieving a urological stone may employ a relatively large diameter endoscope (the ‘mother endoscope’) to gain access to and visualize smaller tracts such as those of the biliary system. In such an example, the mother endoscope may be a duodenoscope. Then, a smaller endoscope, such as a cholangioscope (the ‘daughter endoscope’), may be passed through a lumen of the mother endoscope's guide tube, ultimately extending out of the mother endoscope for advancement into smaller bodily tracts for visualization and operation.

Typically, multi-endoscope procedures require more than one operator to accomplish the required procedure. For example, one operator guides the mother endoscope to the required position and holds it in place, and then the second operator maneuvers the daughter endoscope through the mother endoscope and into the selected location, such as the biliary tree. Utilizing two operators for this procedure often causes problems. For instance, the operators must constantly coordinate to ensure both endoscopes are in their correct positions. Moreover, involvement of two operators increases the cost and time required to complete the procedure.

Some endoscopic systems attempt to solve this problem through various methods of attaching the handle of the daughter endoscope to the mother endoscope. This configuration allows a single operator to stabilize the assembly with one hand and steer the endoscopes with the other. These techniques, however, suffer from several shortcomings. One such technique uses a strap to hold the two handles together. The strap, however, has a tendency to disengage from the endoscopes during the procedure, increasing procedure time and user frustration. Another technique utilizes a snug-fit sleeve between the mother and daughter handles. Such sleeve attachments, however, are tailored for a particular mother/daughter combination, making them unusable for alternate combinations of differing configurations.

Therefore, there exists a need for a system that couples a mother device and daughter device during a procedure. Moreover, there exists a need for a system that may be reused or applied to mother-daughter devices of any geometry without alterations.

### SUMMARY

Embodiments of the present disclosure are directed to systems and methods for coupling multiple endoscopic devices.

One embodiment of the present disclosure is directed towards a device for releasable coupling of a first medical tool to a second medical tool. The device includes an elongate member having a first end and a second end. The first end is configured to be secured to a portion of the first medical tool, and the second end is secured to a coupling sleeve. The coupling sleeve defines a lumen for receiving a portion of the second medical tool.

In various embodiments, the device may include one or more of the following additional features: the coupling sleeve includes a shell; a surface of the sleeve may include an expandable member; the expandable member may be secured to an internal surface of the shell; the expandable member may be secured to an external surface of the sleeve; the expandable member may be an inflatable balloon; the expandable member may further include a valve for inflating the balloon; the expandable member may include a foam structure; the portion of the first medical tool may be a handle; and the portion of the second medical tool may be a handle.

Another embodiment of the present disclosure is directed to a medical system including a first medical tool and a second medical tool. The first medical tool including a first elongate member having a first proximal end, a first distal end, and at least one lumen extending there between. The proximal end of the first medical device includes a handle. The second medical tool including a second elongate member having a second proximal end and a second distal end. The second elongate member is configured to be received in the at least one lumen of the first elongate member. The system further includes a coupling device, having a first end and a second end, for releasably securing a portion of the first medical tool to a portion of the second medical tool. The first end of the coupling device is configured to be secured to the portion of the first medical tool, and the second end is secured to a coupling sleeve, which defines a lumen there through for receiving a portion of the second medical tool.

In various embodiments, the system may include one or more of the following additional features: the coupling sleeve includes a shell; a surface of the sleeve includes an expandable member; the expandable member may be secured to an internal surface of the sleeve; the expandable member may be secured to an internal surface of the shell; the expandable member may be an inflatable balloon; the expandable member may further include a valve for inflating the balloon; the portion of the first medical tool may be the handle; and the portion of the second medical tool is the handle.

A further aspect of the present disclosure may include a method for releasable coupling a first medical tool to a second medical tool disposed within a lumen of the first medical tool. The method includes positioning a proximal portion of the first medical tool within a lumen of a coupling sleeve extending from a proximal portion of the second medical tool. Further, the method includes transitioning an expandable member from a collapsed state to an expanded state. When in the expanded state, the expandable member frictionally retains the position of the first medical tool relative to the coupling sleeve.

In various embodiments, the method further includes inflating the expandable member through a valve.

Additional objects and advantages of the claimed invention will be set forth in part in the description, which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiments of the disclosure, and together with the description, serve to explain the principles of the disclosure.

FIG. 1 is a perspective view of an endoscopy system according to an embodiment of the present disclosure.

FIG. 2 is a perspective view of an attachment system according to an embodiment of the present disclosure.

FIGS. 3A, 3B, 3C, and 3D are cross-sectional views of the system shown in FIG. 2, taken along plane A-A′.

FIGS. 4A and 4B are cross sectional views of an embodiment of the present disclosure.

FIGS. 5A, 5B, and 5C are cross sectional views of a system shown in FIG. 1, taken along plane B-B′.

FIGS. 6A, 6B, and 6C are cross sectional views of a system shown in FIG. 1, taken along plane C-C′.

### DETAILED DESCRIPTION

Reference will now be made in detail to embodiments of the present disclosure, an example of which is illustrated in the accompanying drawings. Wherever possible, the same reference numbers will be used throughout the drawings to refer to the same or like parts.

### Overview

Embodiments of the present disclosure are directed to a universal attachment sleeve that can securely hold a mother-daughter device arrangement in position to enable simultaneous control of the mother and daughter devices by a single operator. The two devices may be secured in a reversible manner such that they can be separated after use and are usable in a standalone manner. To this end, the present disclosure provides a universal attachment sleeve to enable custom fitting of all combinations of a mother device with one or more daughter devices. This structure may enable a secure attachment of the two devices, thereby freeing their handles for easy manipulation with one hand, without any obstruction from straps, struts, or frames, which are conventionally used to secure such devices to one another.

As used herein, the term “mother device” refers to any tubular medical device such as an endoscope that may be used to insert other endoscopic devices into a patient's body. The term “daughter device” refers to a medical device that may be inserted through a mother device, such as endoscopes, fiber optic cables, tissue graspers, morcellators, and other suitable devices for introduction or tissue manipulation. Typically, the insertion tube of the daughter endoscope has a smaller outer diameter than the inner diameter of the mother endoscope. Moreover, throughout this disclosure, the term “distal” refers to the end farthest away from a medical professional when introducing a device in a patient. By contrast, “proximal” refers to the end closest to the medical professional (usually the handle) when placing a device in the patient.

Embodiments of the present disclosure can be used in connection with a variety of endoscopes or suitable introduction sheaths, including cholangioscopes, duodenoscopes, ureterscopes, colonoscopes, and the like. Although exemplary embodiments of the present disclosure are described with reference to attachment of a daughter endoscope with a mother endoscope, specifically the attachment of a duodenoscope (mother endoscope) to a cholangioscope (daughter endoscope), it will be appreciated that aspects of the present disclosure may have wider application. The specifically disclosed devices and techniques may be suitable for use with other medical devices, where an attachment of a device passed through a channel of another device is desired. Accordingly, the following descriptions and illustrations should be considered illustrative in nature, not limiting the scope of the claimed invention.

In addition, while the discussion of systems and methods below may generally refer to “surgical tools,” “surgery,” or a “surgical site” for convenience, the described systems and their methods of use are not limited to procedures that could be considered “surgical,” such as tissue resection and repair. In particular, the described systems can be highly useful for inspection and diagnosis, in addition, or as an alternative to surgery. Moreover, the systems described herein can perform non-medical applications such as the inspection and repair of machinery.

### Exemplary Embodiments

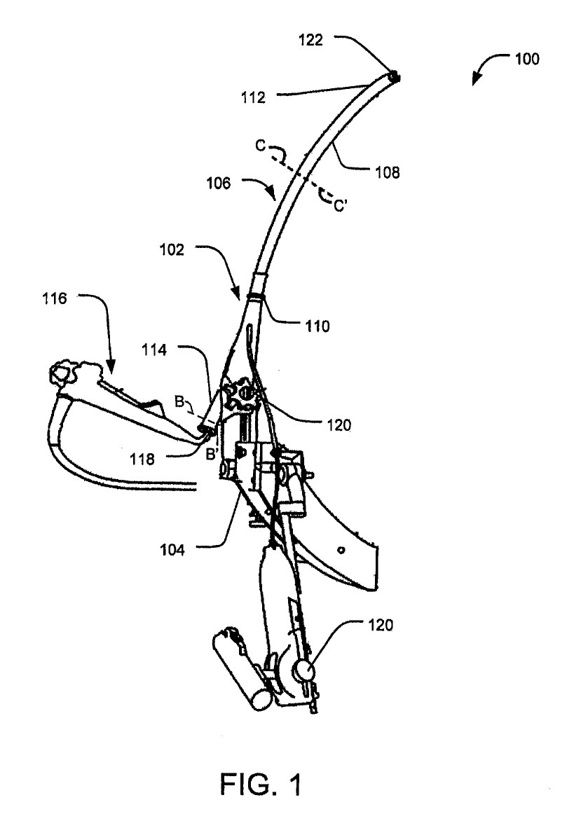

FIG. 1 provides a perspective view of an exemplary system 100 for performing an endoscopic procedure. System 100 includes a mother device 102 having a handle 104 and a guide tube 106. The guide tube 106 may feature an elongate body 108 having a proximal end 110 and distal end 112, and one or more tubular channels or lumens (not shown) may extend throughout its length. The proximal end 110 defines at least one proximal end opening (hereafter port 114), which may receive a daughter device 116.

Similarly, daughter device 116 includes an elongate insertion tube 118 having proximal and distal ends. The daughter device 116 may be inserted through port 114 to enter the guide tube 106 through any of the channels disposed within it. Port 114 is often but not necessarily situated distally of handle 104 on the mother device 102. The port 114 may also be located at the proximal end 110 of the mother endoscope handle 104. Control mechanisms 120 may actuate the distal end 112 of the mother device 102. Further, the distal end 112 defines at least one distal end opening 122 for introducing the elongate member 118 of the daughter device 116 to a target site within a patient.

As noted above, guide tube 106 may define one or more channels adapted to guide at least one elongate tool, such as, e.g., daughter device 116, to a surgical site. In general, the channels comprise hollow lumens that extend between proximal end 110 and distal end 112 of guide tube 106. The number of channels and their configuration varies depending on the intended use of system 100 and the tools required during a procedure. For example, guide tube 106 can include a single channel adapted to receive multiple daughter devices or multiple channels for multiple daughter devices.

Further, the channels of tube 106 may have any suitable cross-sectional configuration. Each channel may have a substantially circular cross-sectional configuration, or in some embodiments, one or more channels may have a cross-sectional configuration that corresponds to a configuration of a particular daughter device 116.

The guide tube 106 may allow passage of one or more daughter devices through any of the channels to perform a desired procedure. The daughter device 116 may include tools such as optical devices including endoscopes, as well as other tools such as jaws, hollow tubes, snares, forceps, needles, or similar tissue manipulation instruments known in the art. The mother and daughter device combinations may include a keyed (e.g., a groove receiving a corresponding tab) interaction.

To ensure single user operation of a mother/daughter combination, the mother endoscope 102 must be secured to the daughter device 116. Specifically, locking the handles, or some other portions, of the two instruments allows a user to hold the entire assembly with one hand and perform the desired operation with the other. To this end, embodiments of the present disclosure employ a sleeve, which may be disposed at various locations (or interfaces) between the mother and daughter devices to securely attach them. The sleeve may be a self-expandable device or may expand by some actuation mechanism. Typically, the sleeve changes state from a collapsed state to an expanded state. The sleeve may transition between the collapsed state and the expanded state using any known expansion or compression mechanism, including, but not limited to, those described later in the disclosure. In each instance, the final configuration exerts a force on the daughter device 116 or mother device 102, frictionally maintaining the two devices in position leaving their handles free for unrestricted manipulation. In some embodiments, mother and daughter devices may be connected via luer-lock connection.

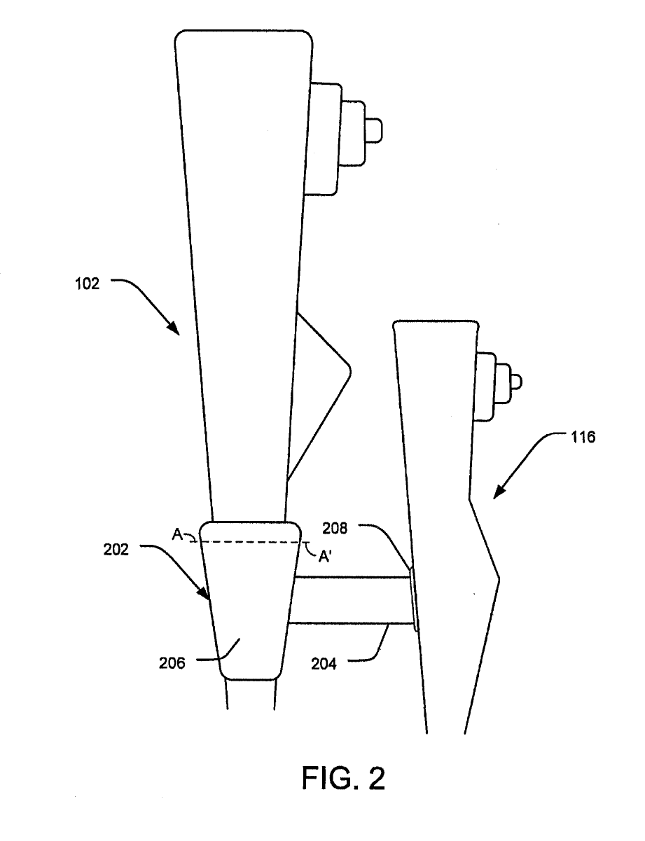

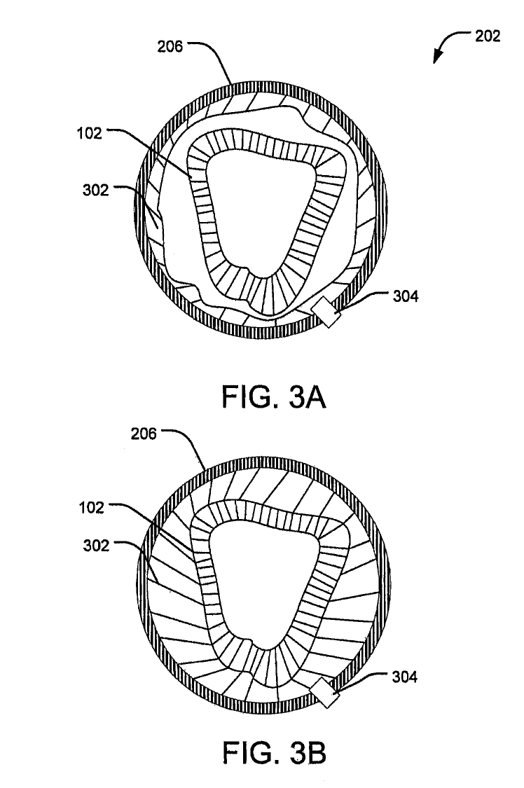

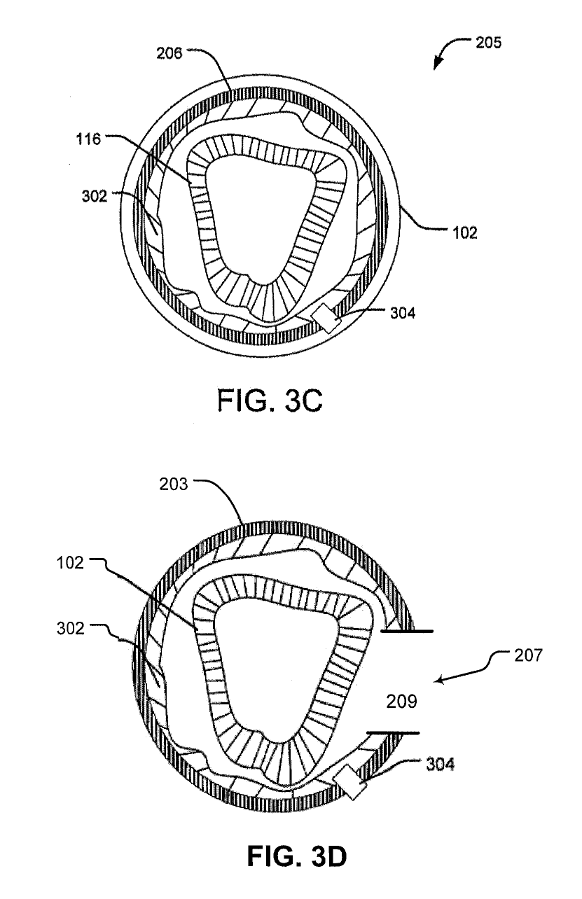

FIG. 2 illustrates a mechanism to securely attach the mother device 102 to the daughter device 116, which may include another endoscope or other suitable introduction sheath, as explained. To this end, a hollow sleeve 202 is secured to the handle of the daughter device 116 in a releasable and reusable manner via, for example, arm 204, which will be discussed in greater detail below. The sleeve 202, as shown in FIG. 2, may include a rigid outer surface (hereafter shell 206) in a form suitable to accommodate the mother endoscope 102. The outer shell 206 may provide a definite shape to the sleeve, such as cylindrical or conical. In some embodiments, the shell 206 may be C-shaped and not tubular. That is, the shell 206 may be snapped over a device handle, for example, rather than rather than having the device handle inserted into the shell longitudinally, see FIG. 3D. An expandable material (not shown in FIG. 2) may line the inner surface of the shell 206. When the mother endoscope is inserted through the lumen of the sleeve, the expandable lining may be configured to selectively expand to fill the entire space between the shell 206 and the device, prohibiting relative movement between the two devices.

Further, arm 204 may extend from the shell 206 to secure the shell 206 to a portion of the daughter device 116. The arm 204 may also be releasably secured to the sleeve 202 and daughter device 116. A sliding lock 208 may secure the arm 204 to the daughter device 116. Alternatively, the shell may be a clamshell housing which snaps around the device. A balloon may line each half of the clamshell or a cylindrical balloon may fit around the daughter device 116 with the clamshell snapped around it. Alternate locking means may include pressure fit, magnetic fit, suction fit, Velcro™, or any suitable attachment means. In some embodiments, the inside surface of shell 206 may be formed of a material with a high-coefficient of friction. Either of the device surfaces may include a layer or coating of high-friction material.

In an alternate embodiment, the arm 204 may be an independent unit attached either to the sleeve 202 and/or to daughter device 116. The arm 204 may thus have attachment means at both ends to secure itself to the sleeve 202 and daughter device 116. In a further embodiment, both ends of the arm 204 may be attached to two substantially similar sleeves (not shown), where one sleeve accommodates the daughter device 116 and the other secures the mother device 102. For example, arm 204 may include a key arrangement, with a tab formed to fit into a receiving groove, or similar known means, for securing the devices. In another embodiment, arm 204 may be a bent wire with attachment means. In some embodiments, arm 204 may include multiple arms with attachment means for securing mother and daughter device. Arm 204 may be a single unit connected across the full length of the sleeves including devices. Secure attachment of any two devices of suitable dimensions may be therefore possible. The sleeve 202, as described in this embodiment, may have a rigid outer shell 206 and an expandable material (not shown) lining the inner surface of the sleeve 202. Alternatively, one or both of two sleeves 202 may be integrally connected to one or both of the mother and daughter devices 102, 116. In addition, one or both of sleeves 202 may be a form-fitting sleeve 202 thereby may not include the expandable inner lining. In one embodiment, the sleeve 202, which slips over the daughter device 116, may be form fit to the shape of the daughter device 116. This enables attachment of a specific daughter device to any mother device. In alternate embodiments, one or both sleeves may include outer shells, outer shell with expandable members, or only expandable members. The outer shell may be formed as a clam shell housing that snaps around the device. Expandable material (e.g., a balloon) may line each half of the claim shell housing, or a cylindrical expandable member may surround the device, with the clam shell housing fitting around the expandable member.

The arm 204 may be made of rigid materials such as ceramics, metals (e.g., stainless steel), and/or hardened plastics. The arm 204 may also be made of semi-rigid materials such as pliable plastics or rubber. In even further embodiments, the arm 204 may be made of super elastic or shape memory materials. The materials may be heat resistant to enable heat sterilization. The arm 204 may also be of any suitable length and cross section, which may also be adjustable. For example, arm 204 may be configured to selectively elongate by, e.g., telescoping between longer and shorter lengths. In a further embodiment, the arm 204 may also be an extension arising from the daughter device 116 to whose distal end a sleeve 202 can be attached. The sleeve 202 may incorporate known locking mechanisms, such as the locking means 208, discussed above, to secure the arm 204 to the mother device 102.

In addition, or alternatively, arm 204 may include multiple arms extending between the mother and daughter devices. For example, in embodiments where both mother and daughter devices are disposed in respective sleeves, multiple arms may extend between the two sleeves. In addition, arm 204 may be a single elongated arm, extending the full length between the respective sleeves. Even further, in embodiments where arm 204 extends from a sleeve, the free end of arm 204 may be formed with, e.g., a peg for mating with a corresponding slot on the other sleeve.

According to the embodiment shown in FIG. 2, sleeve 202 is in communication with daughter device 116 through a detachable arm 204 secured to the daughter device 116 through locking means 208, wherein the arm 204 extends from the rigid shell 206. The sleeve 202 may further be slid over the mother device handle 104 and positioned with respect to the daughter device 116. Once the mother device 102 is aligned with respect to the daughter device 116, the expandable material 302 is inflated to secure the mother device 102 thereby prohibiting relative movement between the two devices.

FIGS. 3A-3C illustrate a cross section of the attachment mechanism to secure the mother device 102. The sleeve 202 may also comprise a valve 304 to enable inflation of the expandable material 302 lining the inner surface of the shell 206. The valve 304 may be present on the periphery of shell 206 or on the inner surface of the shell 206 accessible by any suitable means known in the art, including, e.g., tubing. FIG. 3A illustrates the mother device 102 positioned within the lumen of sleeve 202. Once the mother device 102 is inserted and positioned within the sleeve 202, expandable material 302 may be inflated by means discussed later in the disclosure.

FIG. 3B illustrates the cross section of the sleeve 202 with expandable material 302 in its expanded state. The expandable material 302 expands to grasp the handle 104 securely. Opening the valve 304 may deflate the expandable material 302 by, e.g., letting out the fluid used for expansion. In each of these embodiments, expandable material 302 may extend along full length of a shell, or along a portion of the inner shell surface.

FIG. 3C illustrates a further embodiment 205, in which sleeve 202 is secured to an inner wall of a lumen of mother device 102. The attachment surface of the sleeve 202 is secured to the mother device 102 by convenient means, such as a friction or interference fit, conventional locking mechanism, or the like. After sleeve 202 is attached to mother device 102, a daughter device 116 of suitable dimension may be employed. In the illustrated configuration, daughter device 116 is grasped by expandable material 302 in the same manner as described above.

FIG. 3D presents yet a further embodiment 207, having a C-shaped sleeve 203. An opening 209 lies between the arms of sleeve 203. Sleeve 203 is formed of semi-rigid polymer material capable of flexing at least sufficiently to allow the open ends of the “C” to expand, permitting opening 209 to fit over mother device 102 (or alternatively over daughter device 116) without permanent, plastic deformation of the sleeve 203. Expandable material 302 is attached to the inner surface of sleeve 203, accessed by valve 304, as described above.

In operation, sleeve 203 is snapped over a portion of mother device 102 by flexing the ends of the C-shape and fitting sleeve 203 over the selected portion of mother device 102 (and/or daughter device 116). Mother device 102 is then grasped by expandable material 302 in the same manner set out above. Sleeve 203 then may be used in the same manner as sleeve 202 described above.

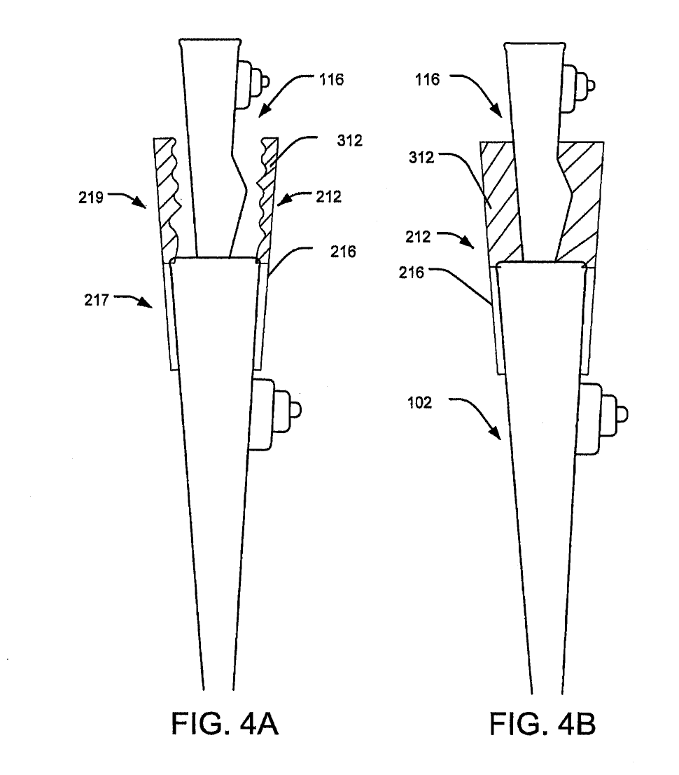

FIGS. 4A-B illustrate yet another embodiment of the present disclosure. Here, rather than securing two endoscopes at points distal to their handles, the handle of the daughter device 116 is secured directly to the handle of the mother device.

As shown in FIG. 4A, a sleeve 212 may be carried on a handle portion of mother device 102, such as, for example, where the port 114 is situated. The sleeve 212 as shown in FIGS. 4A-B may include a rigid outer shell 216, having two portions—a distal portion 217, configured to fit over the proximal end of mother device 102, and a proximal portion 219, extending proximally from the handle portion of mother device 102 and carrying on its inner side an inner lining of expandable material 312. Sleeve 212 may be integrally formed on the handle portion of mother device 102, or it may be configured to be temporarily attached, by a friction or interference fit or other conventional, suitable mechanism.

Daughter device 116 is aligned with the mother device 102, as shown in FIG. 4A, and then the proximal end of the sleeve 212 may be expanded, using expandable material 312, to secure the daughter device 116 as shown in FIG. 4B. As depicted in FIGS. 4A-B, the distal end of the sleeve 202 may be form fit with the mother device handle 104. Proper alignment of the mother and daughter devices includes positioning daughter device 116 to facilitate access to the port 114 of mother device 102.

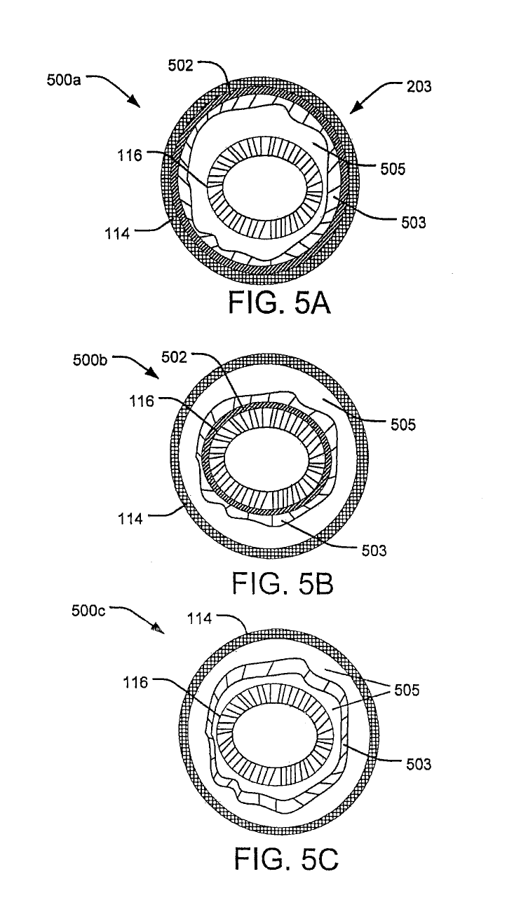

The embodiments discussed above secure the mother and daughter devices by attaching their respective handle portions. FIG. 5A illustrates another embodiment 500a of the present disclosure, in which the endoscopes are secured in the vicinity of port 114. This figure is a cross-sectional view of the system 100 shown in FIG. 1, taken along plane B-B′, immediately distal of port 114. According to this embodiment, a sleeve 502 may be disposed within a port 114 of the mother device 102. The sleeve 502 may be substantially similar to sleeve 202 described above. In practice, the daughter device 116 may include a smaller diameter than is provided within port 114, leaving a gap 505 between an outer surface of daughter device 116 and the inner surface of port 114. To that end, the inner surface of the port 114 is attached to the sleeve 502 having a rigid shell, such as shell 206, which in turn is connected to the expandable member 503. As the daughter device 116 is inserted through the port 114 of the mother device, expandable material 503 expands to fill the entire space between the two devices, as explained in detail below, preventing relative movement between the two devices. In an alternate embodiment, the expandable member 503 may be directly secured to the inner surface of the port 114, and upon activation, the expandable material 503 expands to fill the gap surrounding the daughter device 116.

FIGS. 5B-5C are cross-sectional views of two alternative embodiments 500b, 500c of the system 100 shown in FIG. 1, taken along plane B-B′, immediately distal of port 114. Except for the location of expandable material 503, elements described in connection with FIG. 5A are identical in embodiments 500b and 500c. In the embodiment of 500b, shown in FIG. 5B, sleeve 502 is slid over the daughter device 116, and expandable material 503 is carried on the outer surface of the sleeve 502. Here, the expandable member 503 is only secured to the outer surface of the daughter device 116. The sleeve 502 and daughter device 116 are then inserted into port 114. Embodiment 500c of FIG. 5C, on the other hand, features expandable material 503 carried between the inner walls of port 114 and daughter device 116, not attached to either one. When activated, as discussed below, expandable material 503 expands to fill the gap 505 completely, securing the two endoscopes together. In use, for example, the daughter device 116 may be positioned within port 114 and the expandable material may then be introduced within gap 505.

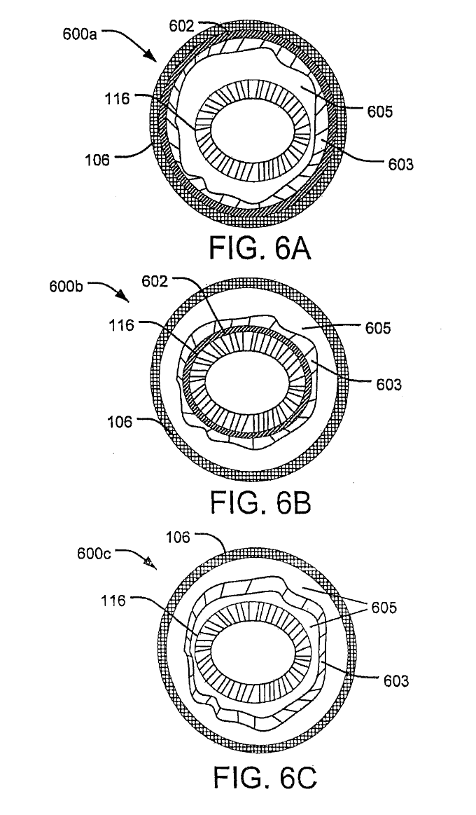

FIGS. 6A-6C present embodiments 600a-600c of the present disclosure, taken on plane C-C′, on tube 106. There, expandable material 603 expands at a location along tube 106, rather than in the vicinity of port 114. As can be seen, apart from that location, the operation and elements of embodiments 600a-600c are identical to those of embodiments 500a-500c discussed above,

The following discussion addresses the expandable material 302, 312, 503, and 603 discussed in connection with embodiments set out above. This material will be referred to herein as “expandable material.” In general, expandable material may be a self-expandable device or may expand by some actuation mechanism. In either case, the expandable material typically changes state from a collapsed state to an expanded state. That transition may be accomplished using any of a number of known mechanisms, discussed in further detail below. In each of the cases, the final configuration exerts a force on the daughter device 116 or mother device 102 to frictionally maintain the two devices in position leaving their handles free for unrestricted manipulation.

In one embodiment, the disclosed expandable material may include a bladder that is inflatable by supplying suitable inflation fluids, such as air or saline, via a valve (see valve 304, FIGS. 3A-3C). Alternatively, the bladder may be a hermetically sealed container, conveniently formed by layers of a flexible material such as fabric, plastic, rubber, or polymers. The layers provide an airtight structure when the edges are sealed. A valve would allow inflation fluid to fill the expandable material using an external supply such as a thumb or motorized pump, or syringe. In one embodiment, the bladder may be a foam structure, such as and open cell foam that absorbs air to expand. The disclosed expandable material may also be deflated to retract the daughter device 116 when required. For deflation purposes, the valve may be a two-way valve made of plastic or metal, which allows air to pass during inflation and deflation.

In an alternate embodiment, the expandable material may be inflatable by using chemical methods. To this end, a mixture of reactants or similar material may be filled in the bladder and once these reactants are exposed to catalysts such as heat, they may produce gases, which expand the expandable material. The inflating medium may also include other fluids such as inert gasses, aqueous liquids, gels or any such suitable media. In each of these embodiments, a safety valve (not shown) may be present in case the pressure of expansion exceeds a threshold limit. In each of these embodiments, the surface of the bladder may have ridges, ribs, bumps, or similar surface roughening for enhanced device attachment.

In an alternate embodiment, the expandable material may take the form of a spring-like devices attached to the surface of the sleeve 202, extending in a direction perpendicular to the longitudinal axis of mother device 102. The spring-like devices would be attached in their compressed state to one side of sleeve 202, and once the daughter device 116 is inserted, the spring may be actuated, e.g., by heat, to extend against daughter device 116, or springs could be actuated by using the device. Various other mechanisms that enable transition of a device from one geometrical state (collapsed) to another (expanded) may replace the mechanisms disclosed here without departing from the scope of the present disclosure. For example, expandable material may be made of distinct longitudinal sections of metal, plastic, or other known rigid material, joined to form a ring-like device. These sections may be joined by rubber, spring, or elastic to form a flexible member, allowing the distinct sections to slide over each other in the collapsed state and abut each other in the expanded state.

In addition, sleeve 202 may assume any suitable configuration such as tubular, ring-shaped, or planar. In the embodiment shown in FIG. 2, sleeve 202 is tubular. It should be understood that the cross-sectional configuration may be adapted according to the gap between the mother and daughter devices 102, 116. In addition, the distance that sleeve 202 extends along the mother device 102 can vary. For example, sleeve 202 may extend over the entire length of the mother device 102, from its proximal to distal end. Alternatively, sleeve 202 may extend along a portion of the length of the mother device 102, or it may be disposed only at certain specified locations on the two devices such as at bends, loops, distal end, and/or the proximal end opening. As shown above, for example, one embodiment shows sleeve 202 positioned at the port 114. Sleeve 202 may be further coated to provide additional properties such as strength, water resistance, heat resistance, or germ resistance. Further, sleeve 202 may also be able to withstand heat sterilization.

For deployment purposes, sleeve 202 may be attached permanently or temporarily to one of mother or daughter device. Alternatively, sleeve 202 may simply slide over daughter device 116. The side of the sleeve 202 forms an attachment surface, which is affixed to the devices to form a permanent or temporary attachment, as desired. Permanent attachment may be accomplished using glues or adhesives, while temporarily attachment may include snap or interference fit, magnetic attachment, or similar mechanisms. The permanent attachment may be employed on certain devices such as endoscopes, which are frequently used in conjunction with multiple devices such as fiber optic cables, elongate tools, tissue manipulators and such similar devices. The temporary attachment or slide-over mechanism enhances re-usability of sleeve 202. In addition, temporarily attached sleeve 202 may be sanitized before use conveniently. Several embodiments of the arrangement of sleeve 202 are shown in FIGS. 5A-5C.

In addition, as shown in FIG. 5C, the disclosed expandable material 503 could be an independent structure, positioned in the gap between mother device 102 and daughter device 116. As shown, the expandable material 503 may slide over the daughter device 116 or may be placed at an appropriate location in mother device's channel. In such implementations, expandable material 503 may be flexible, adapted for flexibly sliding into the concentric gap between the mother and daughter devices 102, 116.

The embodiments set out above are particularly useful in medical procedures involved in treating choledocholithiasis. There, a cholangioscope could be an effective treatment for the management of this disease, but that technique has been limited by the need for two expert operators. The single-operator technique, made possible by the embodiments of the present disclosure, allows simultaneous control of both the cholangioscope (which for the purposes of this disclosure may be the “daughter” device) and duodenoscope (which for the purposes of this disclosure may be the “mother” device). In a cholangioscopic procedure, the duodenoscope is initially inserted into the patient through a natural opening (e.g., the mouth) and is advanced into the duodenum, generally to a point where the bile ducts enter the gall bladder. There, the size of the duodenoscope prevents further access into the biliary system. In accordance with the method set out herein, the duodenoscope can be supplemented with a cholangioscope or any other suitable devices. This smaller instrument is inserted through the lumen of the duodenoscope and extends out of the duodenoscope to enter the biliary vessels.

In some embodiments of the present disclosure, the disclosed sleeve (e.g., sleeve 206) may only include an expandable member and no outer shell. In other embodiments, both sleeves (i.e., sleeves for the mother and daughter devices) may include both expandable members and outer shells.

Embodiments of the present disclosure may be used in various medical or non-medical procedures. In addition, certain aspects of these embodiments may be combined with other aspects of the embodiments, or removed, without departing from the scope of the disclosure.

Other embodiments of the present disclosure will be apparent to those skilled in the art from consideration of the specification and practice of the embodiments disclosed herein. It is intended that the specification and examples be considered as exemplary only, with a true scope and spirit of the present disclosure being indicated by the following claims.

What is claimed is:

1. A device for releasably coupling a first medical tool to a second medical tool, the device comprising:
an elongate member having a first end and a second end, wherein the first end is configured to be secured to a portion of the first medical tool, and the second end is secured to a coupling sleeve, wherein the coupling sleeve defines a lumen therethrough for receiving a portion of the second medical tool.

2. The device of claim 1, wherein a surface of the coupling sleeve includes an expandable member.

3. The device of claim 2, wherein the coupling sleeve includes a shell.

4. The device of claim 2, wherein the expandable member is secured to an internal surface of the coupling sleeve.

5. The device of claim 3, wherein the expandable member is secured to an internal surface of the shell.

6. The device of claim 2, wherein the expandable member is an inflatable balloon.

7. The device of claim 6, wherein the expandable member further includes a valve for inflating the balloon.

8. The device of claim 2, wherein the expandable member includes a foam structure.

9. The device of claim 1, wherein the portion of the first medical tool is a handle.

10. The device of claim 1, wherein the portion of the second medical tool is a handle.

11. A medical system, comprising:
a first medical tool including a first elongate member having a first proximal end, a first distal end, and at least one lumen extending therebetween, wherein the proximal end includes a handle;
a second medical tool including a second elongate member having a second proximal end and a second distal end, wherein the second elongate member is configured to be received in the at least one lumen of the first elongate member; and
a coupling device for releasably securing a portion of the first medical tool to a portion of the second medical tool, wherein the coupling device comprises:
an elongate member having a first end and a second end, wherein the first end is configured to be secured to the portion of the first medical tool, and the second end is secured to a coupling sleeve, wherein the coupling sleeve defines a lumen therethrough for receiving the portion of the second medical tool.

12. The system of claim 11, wherein a surface of the coupling sleeve includes an expandable member.

13. The system of claim 12, wherein the coupling sleeve includes a shell.

14. The system of claim 12, wherein the expandable member is secured to an internal surface of the coupling sleeve.

15. The system of claim 13, wherein the expandable member is secured to an internal surface of the shell.

16. The system of claim 12, wherein the expandable member is an inflatable balloon.

17. The system of claim 16, wherein the expandable member further includes a valve for inflating the balloon.

18. The system of claim 11, wherein the portion of the first medical tool is the handle.

19. The system of claim 11, wherein the portion of the second medical tool is the handle.

20. A method of releasably coupling a first medical tool to a second medical tool disposed within a lumen of the first medical tool, the method comprising:
positioning a proximal portion of the first medical tool within a lumen of a coupling sleeve extending from a proximal portion of the second medical tool; and
transitioning an expandable member from a collapsed state to an expanded state, wherein, when in the expanded state, the expandable member frictionally retains the position of the first medical tool relative to the coupling sleeve.
